(19) United States
(12) Patent Application Publication
(10) Pub. No.: US 2015/0250461 A1
(43) Pub. Date: Sep. 10, 2015

(54) CONSTRICTOR FOR CLOSING OR NARROWING A PASSAGE THROUGH TISSUE OF A HOLLOW ORGAN

(71) Applicant: Eric BERREKLOUW, (US)

(72) Inventor: Eric Berreklouw, Son (NL)

(21) Appl. No.: 14/431,076

(22) PCT Filed: Sep. 25, 2013
(86) PCT No.: PCT/NL2013/050686

§ 371 Date: Mar. 25, 2015

(30) Foreign Application Priority Data
Sep. 26, 2012 (NL) 2009518

(51) Int. Cl. A61B 17/00; A61F 2/24; A61B 17/064

(52) U.S. Cl. CPC A61B 17/0057; A61B 17/064; A61F 2/2442; A61B 2017/00584; A61B 2017/00592; A61B 2017/00637; A61B 2017/00668; A61B 2017/00867; A61B 2017/0641

(57) ABSTRACT

A constrictor for closing or constricting a passage through tissue of a hollow organ, includes: a ring having a thread-like body, which, viewed in the peripheral direction of the ring, extends in a wavy pattern; pins for fastening to tissue surrounding the passage to be closed or restricted; which pins are distributed over the periphery of the ring. Each pin has a fixed end rigidly attached to the ring, and a free pointed end. The constrictor is deformable from a first state into a second state, while a pretension is built up such that the constrictor, in the second state, is under a pretension acting in the direction of the first state. The pretension includes a torsional stress present in portions of the body at the fixed end of each pin, and inclined to want to pivot the respective pin back in the direction of the first state.

The present invention relates to the medical field.

The present invention relates to a medical constrictor for closing or constricting a passage through tissue of a hollow organ, such as the heart or a blood vessel. The constrictor according to the invention comprises a ring consisting of a thread-like body, which, viewed in the peripheral direction of the ring, extends in a wavy pattern; and pins for fastening to tissue surrounding the passage to be closed or constricted, which pins are arranged distributed over the periphery of the ring. In the constrictor according to the invention, each pin has a fixed end, which is rigidly attached to the ring, and a free end, which is of pointed design. Furthermore, the constrictor according to the invention is designed to be deformable from a first state into a second state in a manner that in the meantime—during or as a result of the deformation—a pretension is built up in the constrictor such that the constrictor, in the second state, is under a pretension acting in the direction of the first state.

The constrictor according to the invention is thus reversibly deformable from the first state into the second state in the sense that, upon this deformation, a pretension which acts against the deformation is generated in the constrictor, which pretension—if it is released—will make the constrictor revert to the first state. The return from the second state to the first state thus takes place, as it were, automatically, by making use of the pretension built up in the constrictor.

According to the invention, such a pretension can be realized in a variety of ways, such as by making use of materials, known to the person skilled in the art, which have memory characteristics, referred to as shape memory materials. The necessary shape memory metals/metal alloys denoted by the term ‘shape memory alloy’—such as a NiTi alloy, a CuZiAl alloy or a Ni₂MnGa alloy—as well as plastics—such as shape memory polymers—having such memory characteristics, which can also be used in the constrictor according to the invention, are known to the person skilled in the art. Such shape memory materials can be deformed from a certain initial configuration—in this case the first state—into a second configuration—in this case the second state—and can be frozen, as it were, in the second configuration. No mechanical aids are in this case necessary to maintain the frozen second configuration. The, as it were, ‘frozen’ state can subsequently be lifted (released) by activating the memory effect, after which the material reverts to the initial configuration. Activation of the memory effect can be realized, inter alia, by heating the constrictor to above a certain threshold temperature (or possibly cooling it to below a certain threshold temperature) or—in the case of, for example, a (ferro)magnetic shape memory alloy—by subjecting the constrictor to a magnetic field. Upon activation, the pretension present in the shape memory material, which pretension has been built up during the earlier deformation—is then released in a manner comparable with that of the removal of a physical obstruction in the case of a pretensioned material of spring-steel-like resilience.

In a NiTi alloy, in practice often referred to as nitinol, the conversion from the initial configuration to the second configuration generally takes place at a lowered temperature, for example by placing the material in ice water, so that the material becomes “limp” and can be actively deformed. The ‘freezing’ then takes place by lowering the initial temperature to below a first threshold value and subsequently to below a second threshold value. This is termed an S-shaped temperature-force or temperature-shape curve of the material, wherein the deformation of the material under the influence of the temperature change follows the path of an S-shaped line. The lifting of the frozen state is usually realized by raising the temperature again to above, in the first instance, the second threshold value, and subsequently back above the first threshold value, whereupon the initial configuration is regained. In medical applications of nitinol, the first threshold value often lies a few degrees below the normal body temperature of 37 degrees Celsius, so that the material has, in any event at body temperature, the initial configuration. The first and second threshold value can lie close together, but in practice often lie about 10 degrees Celsius apart. In the case of nitinol, the second threshold value generally lies below or around room temperature. In order then to prevent premature release from the ‘frozen state’ under the influence of the room or body temperature of the patient, it will in practice—and also in accordance with the invention—generally involve an additional mechanically removable obstruction or a cooling system in order to be able to maintain a lower temperature.

Moreover, it should be noted that, in accordance with the invention, the pretension which builds up during deformation from the first into the second state is not only achievable by making use of shape memory materials. Such a pretension can also be generated by making use of an ‘ordinary’ resilient material—of spring-steel-like resilience—which must be kept in a resiliently pretensioned state by means of an external mechanical, removable obstruction.

### DESCRIPTION OF THE PRIOR ART

The older, not yet published patent application PCT/NL2011/050202 filed on 23 Mar. 2011, relates, as the 2nd. aspect, to an annular prosthesis. In PCT/NL2011/050202, it is described that this annular prosthesis can be used to constrict the inlet passage of a heart valve, as well as a closing system for closing an access port, made in a human or animal organ, through the cardiac wall. The annular prosthesis according to PCT/NL2011/050202 consists of a ring with anchorage members, which can be pins. The ring is formed of a thread-like body, which extends in the peripheral direction of the ring in a wavy pattern. The diameter of the ring can here be constricted from one state into another state by virtue of the fact that the wavelengths of the waves of the wavy pattern can be reduced under the influence of a pretension which is inclined to want to constrict the ring. In the embodiments shown in PCT/NL2011/050202, the wavy pattern lies either virtually in the radial plane in both the first and the second state or virtually in a cylindrical plane in both the first and the second state. In both cases, the pins run substantially in the axial direction. PCT/NL2011/050202 further describes that a portion of the pins, at least, is pretensioned in order to, after release of the pretension, displace the free ends of the pins with respect to those ends of the pins which are fastened to the ring. According to PCT/NL2011/050202, this displacement can be in the radially inward direction. In the embodiment in which the wavy pattern of the ring lies virtually in the radial plane, it is a matter of a slight slanting with respect to the truly radial direction, so that the ring plane defined by the wave pattern has a slightly inward—towards the axial centre axis—facing inner side and a slightly outward—away from the axial centre line—facing outer side. The pins are provided on the outer side of the ring plane. The same applies to the embodiment in which the wavy area lies virtually in a cylindrical plane. Here too, it is a matter of a radially inwardly facing side of the ring plane and a radially outwardly facing side of the ring plane and the pins are provided on the outer side of the ring plane.

US 2010/0168790 discloses a device for closing a passage in the wall of a blood vessel. This device consists of a ring of a wavy thread-like body and pointed gripping members for engaging on tissue. The pointed gripping members are provided on inwardly facing troughs of the waves of the wave pattern. Not every wave trough is provided with a pointed gripping member. If a peak centre line is defined as the imaginary line on which the peaks of the waves of the wavy pattern lie, a trough centre line is defined as the imaginary line on which the troughs of the waves of the wavy pattern lie, and a ring plane is defined as the plane which connects the trough centre line and peak centre line and in which the wave pattern extends, then the pointed gripping members project from the side edge, formed by the trough centre line, of this ring plane into the ‘extension’ of this ring plane, that is to say the pointed gripping members project neither above the ring plane, nor below the ring plane. In the deployed state, the ring plane and the pointed gripping members are oriented in the radial direction, wherein the gripping members thus lie in the ‘extension’ of the radially oriented ring plane. Before the closing device according to US 2010/0168790 is placed in a patient, it is brought into a cylindrically oriented state, wherein both the ring plane and the pointed gripping members lie in a common cylindrical plane. This closing device thus has to be brought in compact form to that passage in the wall of the blood vessel which is to be closed, in order to close the passage. To this end, the pointed gripping members, around the passage to be closed, are placed against the wall of the blood vessel and, after this, the ring plane is flipped over from the cylindrical state to the radial state associated with the deployed state. Upon this flip-over, the pointed gripping members press the thereby gripped tissue inward, whereby the passage closes.

US 2008/3193475 and US 2012/035630 disclose a same device as the previously discussed US 2010/0168790 and each also have one of the inventors in accordance with the previously discussed US 2010/0168790. The same applies to US 2008/3193475 and US 2012/035630 as has previously been explained with regard to US 2010/0168790. In the deployed state, the ring plane and the pointed gripping members, in US 2008/3193475 and US 2012/035630, are oriented in the radial direction, while the ring plane and the pointed gripping members, prior to and during the placement in the patient, are in a cylindrically oriented state. After the place of destination has been reached, the ring member is flipped over from the cylindrical position to the radial position. The same also applies to the device known from US 2007/0225762. Here too, a flip-over from a cylindrical position (FIG. 5a) to a radial position (FIG. 5c) takes place.

### The Invention

For the purpose of closer characterization of the constrictor according to the invention, a peak centre line, a trough centre line, a ring plane, an axial centre line and a radial direction are defined for the ring. The peak centre line is defined as the imaginary line on which the peaks of the waves of the wavy pattern lie. The trough centre line is defined as the imaginary line on which the troughs of the waves of the wavy pattern lie. The ring plane is here defined as the plane which connects the peak centre line and the trough centre line and in which the wavy pattern extends. The axial centre line extends in the axial direction of the ring, and the radial direction extends transversely to the axial centre line. Furthermore, the ring plane has two mutually opposing plane sides, namely a first side and a second side lying opposite the first side.

The object of the present invention is to provide an improved constrictor for closing or constricting a passage through tissue, such as, for example, tissue of a hollow organ, which constrictor, on the one hand, can reliably and firmly clasp the tissue and, on the other hand, can reliably and forcefully constrict or close the passage.

c1 This object is achieved according to the invention by providing a constrictor for closing or constricting a passage through tissue of a hollow organ, such as the heart or a blood vessel,
wherein the constrictor comprises:
a ring consisting of a thread-like body, which, viewed in the peripheral direction of the ring, extends in a wavy pattern; and
pins for fastening to tissue surrounding the passage to be closed or restricted; which pins are arranged distributed over the periphery of the ring;
   wherein the ring has:
a peak centre line, which is defined as the imaginary line on which the peaks of the waves of the wavy pattern lie;
a trough centre line, which is defined as the imaginary line on which the troughs of the waves of the wavy pattern lie;
a ring plane, which is defined as the plane which connects the peak centre line and trough centre line and in which the wavy pattern extends;
an axial centre line, which extends in the axial direction of the ring; and
a radial direction, which extends transversely to the axial centre line;
   wherein the ring plane has a first side and a second side lying opposite the first side;
   wherein each pin has a fixed end, which is rigidly attached to the ring, and has a free end, which is of pointed design;
   wherein the constrictor is deformable from a first state into a second state, during which deformation a pretension builds up such that the constrictor, in the second state, is under a pretension acting in the direction of the first state; and
   wherein
the pretension, in this second state, comprises a torsional stress which is present in portions of the thread-like body at the fixed end of each pin, which torsional stress is inclined to pivot the respective pin with respect to the ring into that position of the respective pin with respect to the ring which is associated with the first state;
   and/or
the thread-like body, in portions thereof at the fixed end of each pin, is twisted.

The pins, viewed at the place where they are attached to the ring, stand transversely to the thread-like body.

By rigid attachment of the pin to the ring is here understood that, at the site of the junction or connection between the pin and the ring, no movement in the junction/connection is possible. A transverse force applied to the pin in a direction transversely to the wave pattern is thereby—if the pin, at the site of the junction/connection, stands transversely to the ring—perceived inside the ring, at the site of the connection/junction, as a torsion. It is thus not a hinged connection or a kink-permitting connection. The use of this torsion has the effect that the pins are pressed by the ring from the second state into the first state.

As previously explained, the pretension can produce a directly palpable restoring force, such as, for example, in the case of a spring-steel-like resilient material, or a force which is stored in the memory of the material from which the constrictor is produced and which becomes palpable only once the memory is activated. In the figure description, this is explained in still further detail with reference to the so-called ‘first effect’.

Viewed in the first state, the thread-like body, in the portions thereof at the fixed end of each pin, can be twisted. The torsion present in the first state can have the same direction as that in which, in the second state, the torsional force is acting. Upon the deformation of the constrictor from the first into the second state, the thread-like body will then—for the generation of the torsional stress—be able to be twisted oppositely to the direction of the torsion present in the first state.

It is noted that, in none of the previously discussed publications US 2008/3193475, US 2012/035630, US 2008/3193475 and US 2012/035630 is it a matter of a torsional stress and/or torsion which supports the particular device in the flip-over from one state to the other state.

c2 According to a further embodiment of the constrictor according to the invention, the ring plane, in the first state and in the second state, extends in the radial direction; in the first state, the free ends of the pins point in the direction of the axial centre line; and, in the second state, the free ends of the pins point in the axial direction of the ring. Thus—after the constrictor has been placed in a patient and it has been freed to pass from the second state in the direction of the first state—a sort of anchoring and clamping effect is obtained, wherein the ring is clamped firmly against the tissue by the pins and the pins anchor themselves firmly in the tissue. The constrictor will hereby be firmly attached in the tissue, detachment of the constrictor from the tissue is prevented, and the constricting/closing effect is thus improved. The anchoring and clamping effect will hereupon increase in strength as each pin, or at least a portion of each pin, extends radially in greater measure. It is here of advantage, therefore, if one or more of the pins, in the first state, have a portion which extends at an angle less than 45°, such as less than 30°, with respect to the true radial direction.
c3 According to a further embodiment of the constrictor according to the invention, the torsional stress, in the second state, is directed such that it moves the free end of the respective pin in the direction of the axial centre line of the ring. According to another further embodiment of the constrictor according to the invention, the torsional stress, in the second state, is directed such that it moves the free end of the respective pin in a direction away from the axial centre line of the ring. It is also possible for these two further embodiments to be used in combination, so that both torsional stress which, in the second state, moves free ends of one pins towards the axial centre line and torsional stress which, in the second state, moves free ends of (other) pins in a direction away from the axial centre line, are present.

It is noted that in the present patent application torsional stress as well as bending stress are respectively referred to in the singular. It will be simple, however, that these terms should also be read in the plural. This respectively concerns portions of the thread-like body where, inside the body, torsional stresses or bending stresses prevail, a plurality of such portions being respectively apparent in the thread-like body.

c4 According to a further embodiment of the constrictor according to the invention, the constrictor is made of—or at least, the ring and pins comprise—a shape memory material, such as a shape memory elastomer or a shape memory alloy, for example a nickel-titanium alloy. The constrictor according to the invention can thus comprise, in addition to shape memory material, also other materials, such as a coating which can contain, for example, medication.
c5 According to a further embodiment of the constrictor according to the invention, the constrictor is in the first state slack.
c6 According to a further embodiment of the constrictor according to the invention, the pins are attached to:
the, with respect to the ring, inwardly facing troughs of the waves of the wavy pattern, in particular in the middle of these troughs;
   and/or
the, with respect to the ring, outwardly facing peaks of the waves of the wavy pattern, in particular in the middle of these peaks;
   and/or
flanks of the waves of the wavy pattern, in particular in the middle of these flanks;
   and those portions of the thread-like body which in the second state are under torsional stress are respectively the troughs and/or the peaks and/or the flanks. In particular, the peaks and troughs of the wavy thread-like body lend themselves well to the storage of torsional stresses, since the thread-like body extends in these regions substantially in the peripheral direction of the ring, whilst the pins can here extend substantially transversely to this peripheral direction. In the figure description, this is explained still further with reference to the so-called ‘first effect’.
   c7 According to a further embodiment of the constrictor according to the invention,—viewed and measured from that portion of the axial centre line which is located on the first side of the ring plane—the ring plane angle which the ring plane exhibits with respect to the axial centre line is greater in the second state than in the first state. The effect is thus that, upon the return from the second state in the direction of the first state, the outwardly facing peaks of the wavy pattern of the thread-like body, around the passage to be constricted/closed, are pressed against the tissue and help to push the tissue in the direction of the axial centre line of the ring. This promotes the constricting/closing effect of the constrictor according to the invention. In the figure description, this is explained still further with reference to the so-called ‘third effect’.
   c8 According to a further embodiment of the constrictor according to the invention, the pins, in the first state and in the second state, are located on the first side of the ring plane. The first side of the ring plane is that side of the ring plane which, in the state in which it is placed in the patient, is facing towards the tissue around the passage to be constricted/closed. The pins can thus, in the second state, be easily stuck into the tissue and, following return of the pins in the direction associated with the first state, good anchorage can be achieved in a simple and reliable manner.
   c9 According to a further embodiment of the constrictor according to the invention, the ring is flatter in the second state than in the first state. The constrictor can thus, in the second state, be placed more easily against the tissue around the passage to be constricted/closed.
   c10 According to a further embodiment of the constrictor according to the invention, the diameter of the ring is greater in the second state than in the first state. Upon the return from the second state in the direction of the first state, the ring will thus reduce in diameter and will thus push the pins stuck in the tissue in the direction of the axial centre line. This promotes the constricting/closing effect of the constrictor according to the invention.
   c11 According to a further embodiment of the constrictor according to the invention, the pretension, in this second state, comprises a bending stress which is present in the pins, which bending stress is inclined to bend the respective pin such that the free end of the pin moves towards the centre of the ring. This promotes the constricting/closing effect of the constrictor according to the invention. In the figure description, this is explained still further with reference to the so-called ‘second effect’.
   c12 According to a further embodiment of the constrictor according to the invention, the pins, in the second state, are stretched in the axial direction. This makes it easier to stick the pins into the tissue.
   c13 According to a further embodiment of the constrictor according to the invention, the pretension, in this second state, comprises a bending stress which is present in the flanks of the wavy thread-like body, which bending stress is inclined to bend the respective flank such that the peaks of the wavy pattern move towards the axial centre line. This is explained in greater detail in the figure description with reference to the so-called ‘fourth effect’.
   c14 According to a further embodiment of the constrictor according to the invention, the free ends of the pins, in the first state, lie close to the axial centre line of the ring, such as at a distance of 5 mm or less from the axial centre line. This benefits the closing-off effect of the constrictor according to the invention. After all, if the free ends of the pins, in the first state, lie close to the axial centre line of the ring, these free ends, when the constrictor is placed in a patient, will lie close to the centre of the passage to be closed. The free ends of the pins are thus capable of supporting the tissue in the centre of the passage to be closed, and of preventing this tissue from being pushed away in the axial direction, which could lead to leaking of the closed-off passage.
   c15/16 According to a further embodiment of the constrictor according to the invention, the ring plane extends, in the first state, in the radial direction; and the pins are arc-shaped with an arc angle of at least 20°, such as at least 30° or at least 45°, and extend in a plane transversely to the ring plane. This benefits the anchorage of the pins in the tissue. According to a yet further embodiment hereof, the arc shape of the pins extends, in the first state, over an arc angle of at least 45°, such as at least 60°, or about 90°.
   c17 According to a further embodiment of the constrictor according to the invention having arc-shaped pins, the pins, in the second state, are stretched in an arc shape reduced from the first state and extend in the axial direction. On the one hand, the, in the first state, arc-shaped pins can thus be stuck easily into the tissue when the constrictor is in the second state, whilst, on the other hand, the arc shape of the pins, in the first state, make detachment of the constrictor from the tissue more difficult. If the pretension present in the pins has already been released at the time of insertion in the tissue, the pins will in this embodiment be stuck into the issue along a curved path, which reduces damage to the tissue by the pins.
   c18 According to a further embodiment of the constrictor according to the invention, the ring plane, viewed on this first side and in the first state, extends at a ring plane angle of 30° to 80°, such as 45° to 80° or 45° to 70°, with respect to the axial centre line. The ring plane will thus under varying circumstances, after placement in the patient, generally nestle comfortably against the tissue.
   c19 According to a further embodiment of the constrictor according to the invention, the ring plane, viewed on this first side and in the second state, extends at a ring plane angle with respect to the axial centre line which is at least 10°, such as 15° to 45° or 15° to 30°, greater than the ring plane angle which the ring plane, viewed on the first side and in the first state, exhibits with respect to the axial centre line. In this embodiment, the outwardly directed peaks of the wavy pattern will be able to be pressed firmly against the tissue around the passage to be constricted/closed.
   c20 According to a further embodiment of the constrictor according to the invention, the ring plane, viewed on the first side and in the second state, extends at a ring plane angle of 45° to 120° with respect to the axial centre line.
   c21 According to a further embodiment of the constrictor according to the invention, the ring plane, in particular the first side thereof, has in the first state a conical shape or the shape of a portion of a cylinder surface.
   c22 According to a further embodiment of the constrictor according to the invention, the ring and pins are formed as a complete whole by cutting-out from a single plate, in particular a flat plate; or from a single three-dimensional body. By cutting-out are here understood techniques such as laser-cutting and etching. By cutting out the ring and pins from a single plate or a single body, the pins are already directly rigidly attached to the ring. Separate fastening steps for fastening the pins to the ring, for example by means of welding, are then superfluous.
   c23 According to a further embodiment of the constrictor according to the invention, the thread-like body, and preferably also the pins, have a right-angled cross section. A right-angled cross section of the ring makes it possible to be able to verify from the outside by visual inspection whether the torsional stress has also actually been introduced into the ring when the constrictor is in the second state.

If the pins are produced separate from the ring, according to a further embodiment of the constrictor according to the invention, wherein the thread-like body has a right-angled cross section, the pins can be placed with the end face of the fixed end against a side face of the thread and welded to the thread. The right-angled cross section provides flat side faces, to which a pin can easily be welded.

According to a further embodiment of the constrictor according to the invention, wherein a wave centre line is defined as the imaginary line which interconnects those points of the wave pattern which respectively lie midway between a trough and a neighbouring peak, the wave centre line runs according to a sinusoidal pattern. This sinusoidal pattern will describe, in particular, three wave cycles. A constrictor of this type can be used, inter alia, in connection with the annulus of an aorta valve. The annulus of an aorta valve namely has a sinusoidal shape comprising three cycles.

c24 According to a further embodiment of the constrictor according to the invention, which is designed, in particular, to close a passage, the external diameter of the ring, in the first state, is less than or equal to 30 mm, such as less than or equal to 20 mm.
c26/27 According to a further aspect, the invention relates to an assembly comprising a constrictor according to the invention as well as a medical instrument, wherein the medical instrument comprises a pin-shaped portion, on which the constrictor, currently in the second state, is provided. Such a pin-shaped portion can be hollow or solid. The pin-shaped portion can be cylindrical, for example. Furthermore, the outer periphery of the pin-shaped portion can have a knobbed pattern, whereof each knob fits in a portion of a wave of the wavy pattern, such as an elongated and flattened wave peak of the wavy pattern.
c28 According to yet another aspect, the invention relates to a method for producing a constrictor according to the invention, which method comprises the following steps:
the cutting-out of the pins and the ring consisting of a thread-like body, as a complete whole, from a plate of a shape memory alloy, wherein the pins point with their free ends, viewed with respect to the ring, in the radially outward direction;
the bringing of the cut-out ring with pins into a first state, in which the pins lie with their free ends on the first side of the ring plane, point in the direction of the axial centre line and extend in a plane transversely to the ring plane defined by the ring; and
the subjection of the constrictor, currently in a first state, to a temperature treatment, such that this first state is stored in the memory of the shape memory alloy.

The fact that the pins point in the radially outward direction during the cut-out operation enables the length of the pins to be essentially unrestrictedly large. The pins can thus be longer than the radial distance of the fixed end of the pin to the centre of the ring. This provides a wide degree of scope in shaping of the pins, since they can have any length according to requirement.

c29 According to a further embodiment of the method for producing the constrictor according to the invention, the step of bringing the ring with pins into a first state comprises: the pivoting of the pins into a position in which the free ends thereof point towards the axial centre line of the ring, such that the thread-like body at the fixed end of each respective pin twists.
c30 According to a further embodiment of the method for producing the constrictor according to the invention, the step of bringing the ring with pins into a first state comprises: the curving of the pins into an arc shape with an arc angle of at least 30°.
c31/32 According to a further embodiment of the method for producing the constrictor according to the invention, the wavy pattern is formed, during the cutting-out step, by cutting out the ring from the plate in accordance with that wave pattern. Alternatively, it is also possible for the wavy pattern to be formed, during the step of bringing into the first state, by deforming the thread-like ring into the wavy pattern.
c33 According to a further embodiment of the method for producing the constrictor according to the invention, the wavy pattern of the ring is formed, during the step of bringing into the first state, into a conically shaped structure or a structure having the shape of a portion of a cylinder.
c34 According to yet another aspect, the invention relates to a method for preparing for use a constrictor according to the invention, wherein the constrictor is brought from the first state into the second state by pivoting the pins such that in the ring, at the fixed end of each pin, a torsional stress builds up, which torsional stress acts in a direction with the intent of pivoting the pin with respect to the ring back in the direction of that position of the respective pin which is associated with the first state.
c35 According to a further embodiment of the method for preparing for use, the pins, in the conversion from the first state into the second state, are pivoted into a position which is oriented axially with respect to the ring.
c36 According to a further embodiment of the method for preparing for use, the pins, when the constrictor is brought from the first state into the second state, are bent, so that in each pin a bending stress builds up, which bending stress acts with the intent of bending the pin back into that shape of the respective pin which is associated with the first state.
c37 According to a further embodiment of the method for preparing for use, when the constrictor is brought from the first state into the second state, the ring plane angle which the ring plane exhibits with respect to the axial centre line is changed.
c38 According to a further embodiment of the method for preparing for use, when the constrictor is brought from the first state into the second state, the ring plane angle which the ring plane exhibits with respect to the axial centre line changes as a result of the pivoting of the pins.
c39 According to a further embodiment of the method for preparing for use, when the constrictor is brought from the first state into the second state, and viewed in the radial direction of the ring plane, that curvature of the ring plane which the ring plane exhibits with respect to the axial centre line is changed.
c40 According to a further embodiment of the method for preparing for use, when the constrictor is brought from the first state into the second state, and viewed in the radial direction of the ring plane, that curvature of the ring plane which the ring plane exhibits with respect to the axial centre line changes as a result of the pivoting of the pins.
c41 According to a further embodiment of the method for preparing for use, when the constrictor is brought from the first state into the second state, the diameter of the ring is enlarged.
c42 According to a yet further aspect, the invention relates to a method for placing a constrictor according to the invention in tissue, wherein in a first step, from the second state with enlarged diameter of the ring and with pins stretched in the axial direction, the pins are stuck into the tissue and are released in order to return in the direction of that position of the pins which is associated with the first state, whilst the ring is refrained from reverting to the form associated with the first state;
wherein in a second step the ring is released in order to return towards the form associated with first state; and
wherein the second step takes place at a later point than the first step.
c43/44 According to a yet further aspect, the invention relates to the use of a constrictor according to the invention for closing a passage through a wall of a hollow organ, such as a heart or blood vessel; or for constricting an annulus of a heart valve.

The present invention will be explained in greater detail below with reference to a drawing in which an embodiment is represented. In this drawing:

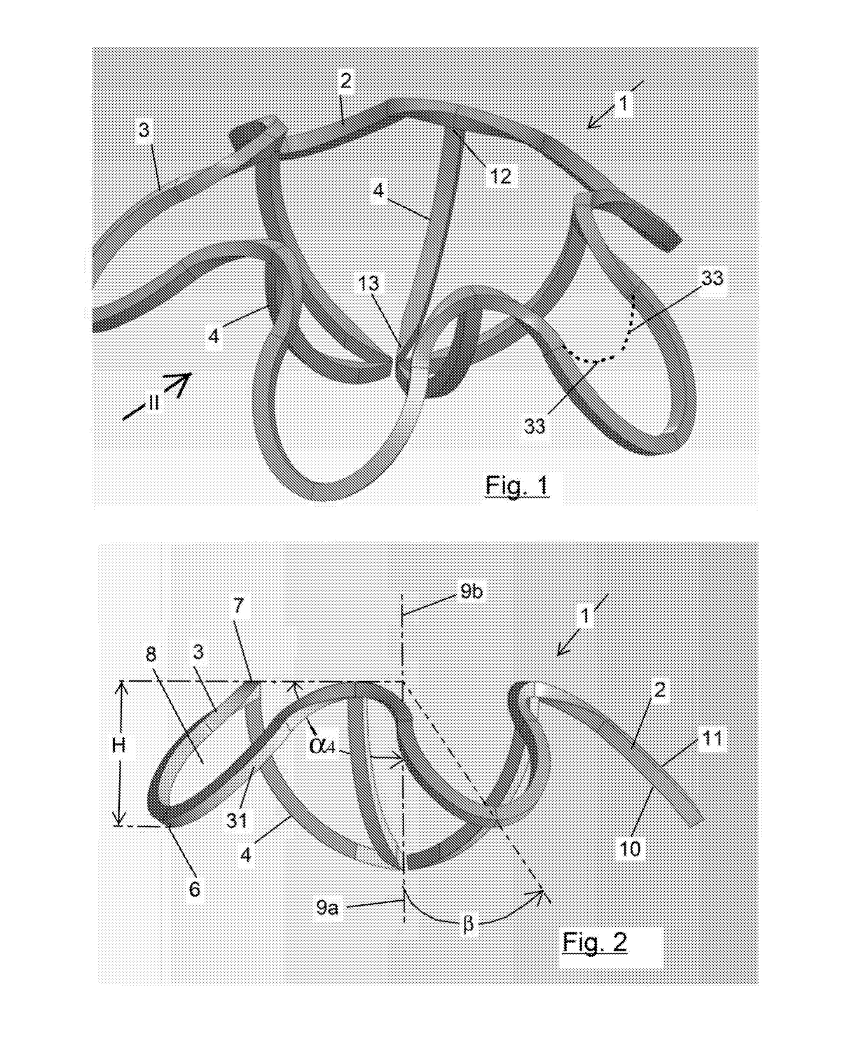

FIG. 1 shows a perspective view, obliquely from above, a first constrictor according to the invention;

FIG. 2 shows a side view, in accordance with arrow II in FIG. 1, of the first constrictor according to FIG. 1, wherein only that half of the first constrictor which is situated on the view side is portrayed in order to keep the drawing simple;

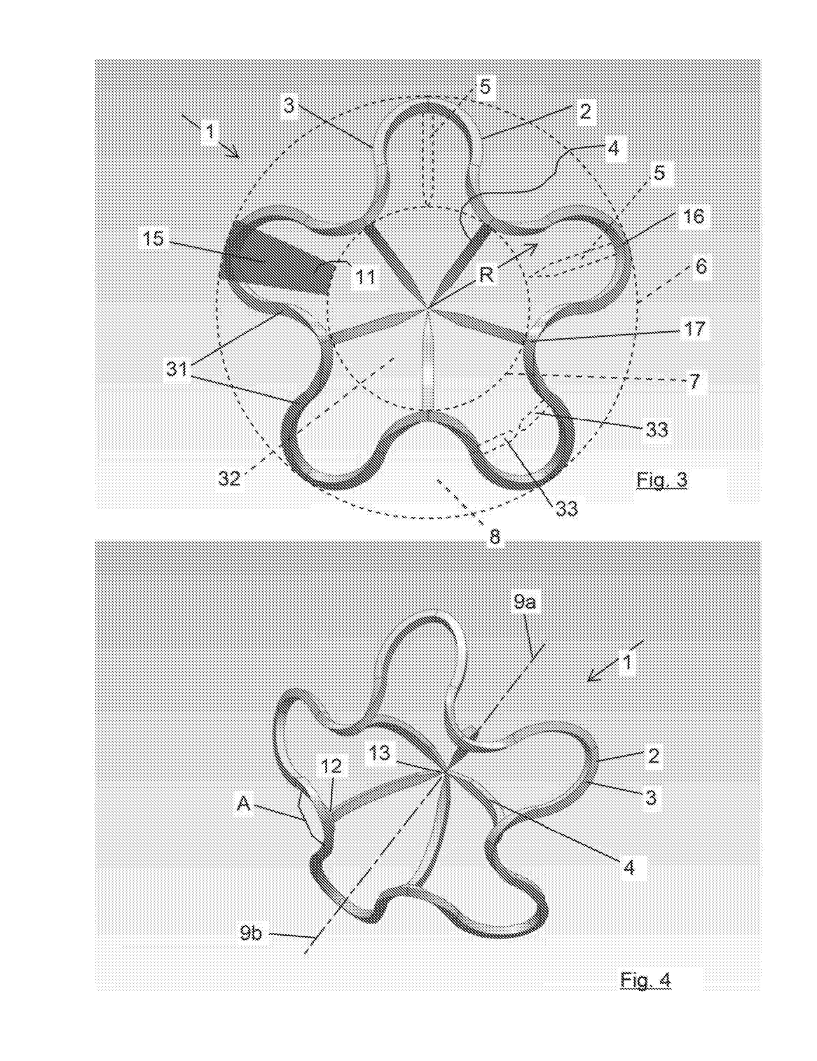

FIG. 3 shows a top view, in accordance with arrow III in FIG. 2, of the first constrictor according to FIGS. 1 and 2;

FIG. 4 shows a perspective view of the first constrictor according to FIGS. 1-3, wherein the constrictor is portrayed tilted;

FIGS. 5-10 show photos illustrating the use of the first constrictor according to FIGS. 1-4;

FIG. 11 shows a perspective view of a second constrictor according to the invention, which is portrayed in accordance with the view of FIG. 1;

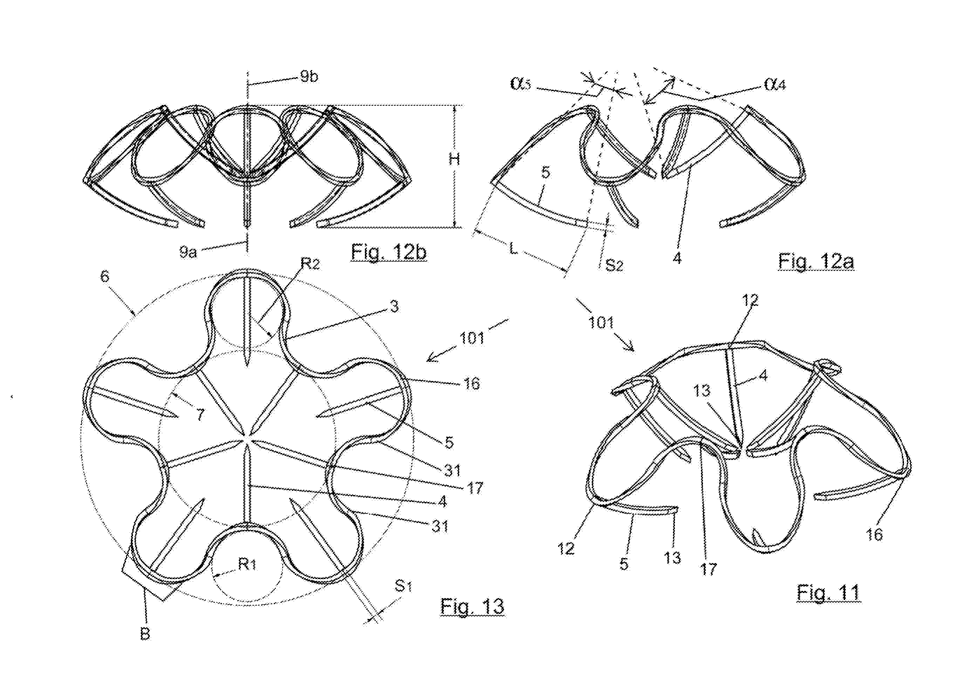

FIG. 12a shows a side view of the second constrictor, which is portrayed in accordance with the view of FIG. 2;

FIG. 12b shows a side view in accordance with FIG. 12a, wherein, however, also the rearmost portion of the constrictor is portrayed;

FIG. 13 shows a top view of the second constrictor, which is portrayed in accordance with the view of FIG. 3; and

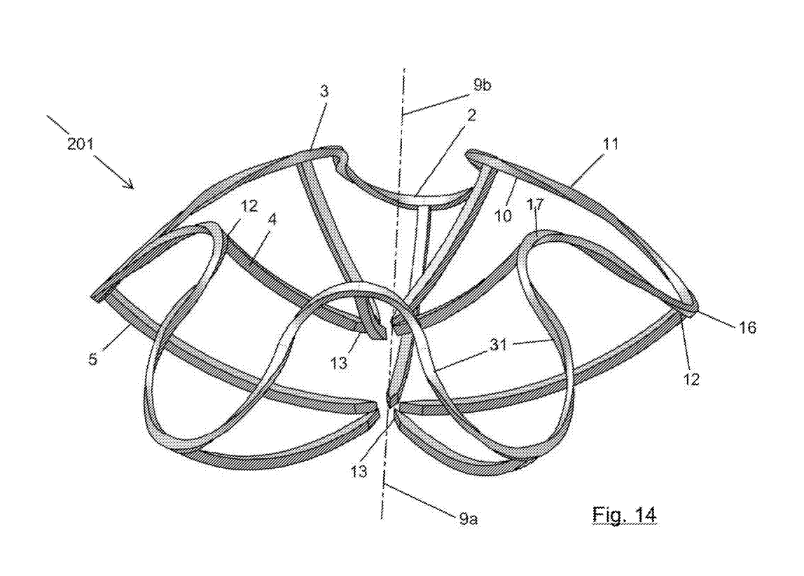

FIG. 14 shows a perspective view of a third constrictor according to the invention.

As indicated above, FIGS. 1-10 show a first constrictor 1 according to the invention, FIGS. 11-13 show a second constrictor 101 according to the invention, and FIG. 14 shows a third constrictor 201 according to the invention. These constrictors will be discussed below with reference to primarily the first constrictor 1 from FIGS. 1-10, with occasionally an excursion to the second constrictor 101 from FIGS. 11-13 and the third constrictor 201 from FIG. 14. In FIGS. 11-13 and FIG. 14, for the second constrictor 101 and the third constrictor 201 respectively, the same reference numbers and letters are used for corresponding items as for the first constrictor 1.

With reference to FIGS. 1-4, it can be seen that the constrictor 1 according to the invention is built up of a ring 2 comprising, in this example, five pins 4 and a wavy pattern of five waves. It should be noted that the constrictor 1 according to the invention can also have more or fewer pins 4, such as three, four, six, seven, eight, nine, ten, eleven, twelve or more pins 4 and/or more or fewer than five waves, such as three, four, six, seven, eight, nine, ten, eleven, twelve, thirteen, fourteen or more waves.

The ring 2 consists of a body 3, which is thread-like and extends in the peripheral direction of the ring 2 in a wavy pattern. The wave pattern has for each wave cycle a peak 16 and a trough 17. In FIGS. 1-4, the peaks 16 are facing outwards with respect to the ring 2 and the troughs 17 are facing inwards with respect to the ring 2. With reference to FIG. 3, the ring has a peak centre line 6 and a trough centre line 7. The peak centre line 6 is the imaginary line which connects the peaks 16 of the wavy pattern, and the trough centre line 7 is the imaginary line which connects the troughs 17 of the wavy pattern. The peak centre line 6 and trough centre line 7 together delimit a ring plane 8 in which the wavy pattern of the thread-like body 3 extends. For illustration of the ring plane 8, in FIG. 3 a sector of the ring plane 8, denoted by 15, is represented transparently darker.

With reference to FIG. 2, the ring plane 8 has two (plane) sides, the first side 10 and the second side 11. The ring 2 further has an axial centre line 9a, 9b, around which the thread-like body 3 extends, and a radial direction R, which stands transversely to the axial centre line 9a, 9b—hereinafter together denoted by 9. The axial centre line 9 has an uppermost portion 9b, which is located on the second side 11 of the ring plane 8, and a lowermost portion 9a, which is located on the first side 10 of the ring plane 8. This lowermost 9a and this uppermost 9b portion of the axial centre line lie in extension one to the other and together form the axial centre line denoted by 9.

In FIG. 2, the surface of the first side 10 of the ring plane 8 is facing obliquely radially towards the lowermost portion 9a of the axial centre line 9, whilst the surface of the second side 11, precisely oppositely, is facing obliquely radially outwards. It can further be seen—see, inter alia, FIG. 2—that the ring plane 8 in this embodiment not only runs obliquely in the radial direction, but also is curved. The first side 10 is here concavely curved and the second side 11 is convexly curved. In a general sense, it is here noted that the ring plane 8, viewed in the first state, can have diverse shapes.

The shape of the ring plane 8 can be tailored to the intended application of the constrictor according to the invention. The ring plane 8 can have a flat 2-dimensional or a more complex 3-dimensional shape. Examples of possible shapes are, inter alia: a (frusto)conical shape, the shape of a portion of a cylinder, such as a semi-cylindrical shape, a saddle shape, and a sine-like or sinusoidal shape. Where the constrictor according to the invention is used to constrict the annulus of a heart valve, the shape of the cylindrical plane, in particular, will be chosen such that this matches the annulus of a—in particular human—mitral valve, aorta valve, tricuspid valve or pulmonary valve. Shapes of these valves and the annulus thereof are extensively described in the literature, see, for example:
Anatomy, mechanics, and pathophysiology of the mitral annulus Jeffrey J. Silbiger, MD Am Heart J. 2012 August; 164(2):163-76 See FIG. 3.
Three-Dimensional Echocardiographic Analysis of Mitral Annular Dynamics Implication for Annuloplasty Selection Melissa M. Levack, MD*; Arminder S. Jassar, MD*; Eric K. Shang, MD; Mathieu Vergnat, MD; Y. Joseph Woo, MD; Michael A. Acker, MD; Benjamin M. Jackson, MD; Joseph H. Gorman III, MD; Robert C. Gorman, MD Circulation. 2012; 126[suppl 1]:[pp. 183-188] See FIG. 1B.
Annular Geometry and Motion in Human Ischemic Mitral Regurgitation: Novel Assessment With Three-Dimensional Echocardiography and Computer Reconstruction Rashid M. Ahmad, MD, A. Marc Gillinov, MD, Patrick M. McCarthy, MD, Eugene H. Blackstone, MD, Carolyn Apperson-Hansen, MS, Jian Xin Qin, MD, Deborah Agler, RCDS, Takahiro Shiota, MD, and Delos M. Cosgrove, MD Ann Thorac Surg 2004; 78:2063-8
Three-dimensional echocardiography in mitral valve disease. Valocik Gabriel, Otto Kamp, and Cees A. Visser. EHJ Cardiovascular Imaging (2005) Volume 6, Issue 6, [pp. 443-454.]
The aortic interleaflet triangles annuloplasty: a multidisciplinary appraisal Andrea Mangini a,c,*, Massimo Giovanni Lemma a,c, Monica Soncini b,c, Emiliano Votta b,c, Monica Contino a,c, Riccardo Vismara b,c, Alberto Redaelli b,c, Carlo Antona a,c. European Journal of Cardio-thoracic Surgery 40 (2011) 851-857

As can be seen in FIGS. 1-4, the ring plane 8 extends—at least in these figures—at a ring plane angle β of about 45° with respect to the axial centre line 9. The ring plane 8 thus extends in the radial direction and extends—at least in FIGS. 1-4—in equal measure also in the axial direction. With reference to FIG. 2, it is noted that the angle β —also referred to as the ring plane angle, which, measured from the axial centre line 9, represents the angle between the portion 9a of the axial centre line 9 and the ring plane 8—can also assume other values, such as essentially any value within the range of 30°-150°. The ring plane angle will in the first state be able to assume a value within a range of, for example, [30°, 90°], or [30°, 80° ], or [30°, 70°], or [45°, 90°], or [45°, 80° ] or [45°, 70° ] or [60°, 90°], or [60°, 80° ]. In the so-called second state, which is to be discussed further on, the ring plane angle β will generally be greater than in the first state. For the second state, the ring plane angle β can be, for example, also greater than 90°, whilst in the first state it is less than 90°. It is provided that, in the second state, the ring plane angle β can be up to and including 150°, such as up to and including 135°, or up to and including 120°.

The pins 4 each have a fixed end 12, and a free end 13 which is pointed. The pins 4 are rigidly attached to the ring 2 by the fixed end 12. By rigidly attached is here understood that the ‘connection’ of pin 4 to ring 2 allows no movement in the ‘connection’, so that a transverse force applied to the pin is felt in the ring as a torsional load. Such a connection can be realized, inter alia, by producing the ring and the pins from one piece of plate or from a 3-dimensional structure, or by fastening the pins 4 to the ring 2 by means of a welding technique.

It is noted that although in FIGS. 1-10 the pins 4 are respectively provided on the inner side of the ring in the golf troughs 17, the pins can also be provided on the outer side on the wave peaks 16. In FIG. 3, this is illustrated for the first constrictor 1 by two pins 5 portrayed in a dashed line, in FIGS. 11-13 this can be seen with reference to the second constrictor 101, and in FIG. 14 this can be seen for the third constrictor 201. Although in FIG. 3 only two pins 5 are shown in order to keep the drawing simple, these can also be greater in number, as portrayed in FIGS. 11-14. In particular, the pins 5—just like the pins 4—will be provided evenly distributed over the periphery of the ring. In addition, each peak 16 will be able to be provided with a pin 5. In FIG. 3, the pins 5 are portrayed as—in the so-called first state—reaching as far as the trough centre line 17, yet the pointed ends of the pins 5 can also reach past the trough centre line 17, as portrayed in FIGS. 10-11, or even as far as the axial centre line 9, as portrayed in FIG. 14. The pins 5 can extend over the same arc angles α₅ as those which are quoted for the pins 4. Finally, it is noted that the pins 5 can also take the place of the pins 4—that is to say, pins 5 but no pins 4. It is also conceivable for intermediate pins to be provided on the ring between the wave troughs and wave peaks, such as in the middle of the flanks 31. Such intermediate pins can be used in combination with the pins 4 and/or 5, but can also be used in place of the pins 4 and/or 5.

Furthermore, it is noted that, in addition to the pins 4 or in addition to the pins 5 or in addition to the pins 4 and 5 or in place of pins 4 and 5, pins 33—2 of these are shown schematically for illustration purposes with dashed lines in FIGS. 1 and 3—can be provided on the flanks 31 of the wavy pattern. In accordance with pins 4 and 5, these pins 33 will also be rigidly attached to the ring 2 by their fixed end 13. Pins 33 have in common with pins 4 and 5 that they, at the site where they are attached to the ring, stand essentially transversely to the, thread-like body 3. Furthermore, the pins 33 can be curved in a manner comparable to the pins 4 and 5. Instead of the pins 33 pointing with their free ends towards the centre of the ring, in this embodiment, in the first state, they will also be able to point in the peripheral direction of the ring. In the first state, they can point, for example, towards one another or point away from one another in pairs.

The pins 33 which point towards one another, as portrayed schematically in FIGS. 1 and 3, when they are seated in tissue, will clasp the tissue firmly in mutual cooperation. With reference to FIGS. 11-14, a comparable effect can also be achieved by fitting the pins 4 the other way round, in the sense that the free ends point radially outwards, instead of inwards as portrayed in FIGS. 11-14. The pins 4 then stay on the same side of the ring plane 8 as they do in FIGS. 1-4. A pattern of, in the first state, alternately inward pointing pins 5 and outward pointing pins 4 is then obtained.

As can be seen in FIGS. 1-4, the pins 4 are arc-shaped. In FIGS. 1-4, the pins 4 extend over—see FIG. 2—an arc angle α₄ of about 90°. This arc angle α₄ can also however have a different value. The pins will be more or less bent, depending on the tissue. Furthermore, the bending of the pins, viewed along the length of the pins, can be equal, but also unequal. For example, the curvature can increase or, indeed, decrease in strength from the fixed end of the pins to the free end of the pins. Thus, in the embodiment according to FIGS. 11-13, the arc angle, see in particular FIG. 12, is about 20° for both the pins 4 and the pins 5, see arc angle α₄ for the pins 4 and arc angle α₅ for the pins 5. In FIG. 14, the arc angle α₄ for the pins 4 is about 20° and the arc angle α₅ for the pins 5 is about 40°. The arc angle α₄, α₅ can, in the first state, also be greater than 90°. In a general sense, it is provided that the arc angle α₄, α₅ can assume values up to 120° to 135°. In the first state, the arc angle α₄, α₅ can thus assume, for example, a value within the range of [20°, 135°], or [20°, 120°], [30°, 135°], or [30°, 120°], [45°, 135°] or [45°, 120°], or [60°, 135°], or [60°, 120°], or [75°, 135° ] or [75°, 120°], or [90°, 120°], or [90°, 135° ]. In a general sense, with respect to the pins 4, as well as with respect to the pins 5, it is noted that these can mutually differ in length, shape and degree of bending/curvature.

The constrictor according to the invention can be produced, inter alia, by cutting this out from a plate of suitable metal, in particular a shape memory alloy such as nitinol. This plate will, in particular, be flat. It is possible, however, to cut out the constrictor from a 3-dimensional structure, such as a block of suitable material or a conical or cylindrical plate. The cutting-out can be realized by means of techniques which are known per se, such as laser cutting or etching (etching′ is here also understood under the term ‘cutting’).

The wavy shape can here be realized directly during the cut-out by cutting out a wavy ring structure from the plate. The wavy shape can also be realized by first cutting a non-wavy or slightly wavy ring out of the plate and subsequently deforming this ring into the desired wave pattern. In the case of nitinol, this generally happens at high temperatures and with the aid of a template, which is generally denoted by the term ‘temperature shape setting’.

The constrictor will in particular be cut out from a flat plate. It is also possible, however, to cut out the constrictor from a curved plate, in which case the curvature of the plate can be such that the ring plane of the cut-out constrictor has the desired position with respect to the axial centre line already directly during the cut-out operation.

During the cut-out operation, the pins 4—and/or, if present, the pins 5—will be facing with the fixed end 12 towards the middle of the ring and be facing with the free end 13 in the radially outward direction with respect to the ring. Where the wavy shape is cut out directly from the plate, pins 4, depending on the length thereof, will lie wholly or largely between the waves of the wave pattern and the pins 5 will lie wholly on the outer side of the wavy pattern. The reverse—that is to say the free ends of the pins 4 and/or 5 point in the radially inward direction during the cut-out—is also possible for the pins 4 and/or 5. However, since the pins 4, 5 then, during the cut-out, point with their free ends towards the centre of the ring, the lengths of the pins will be limited. In case of excessively large lengths, they will hit one another in the centre of the ring. If yet greater lengths are necessary, the pins would have to intersect in the centre, which is impossible when cutting out from a plate.

After the constrictor has been cut out from the plate, the pins 4, 5, if so desired, will be curved, for example to an arc angle α₄, α₅ of about 90° in accordance with FIGS. 1-4 or an arc angle of about 20° in accordance with FIGS. 11-14. Where the pins 4, 5, during the cut-out operation, point radially outwards with their free ends 13, the pins 4, 5 will also be pivoted such that the free ends 13 of the pins 4 end up pointing towards the axial centre line. The ring 2 will hereupon, in the region around the connection of the pins 4 and 5 to the ring 2—thus in the region of the troughs and peaks of the wave pattern—respectively twist.

A constrictor having a form as portrayed in FIGS. 1-4 can thus be obtained. This constrictor is subsequently subjected to a temperature treatment. If a shape memory alloy is used for the constrictor 1, the form in which the constrictor is currently found, so to speak, is thus stored in the memory′ of the shape memory alloy/of the constrictor by means of a so-called ‘temperature shape setting’. In a temperature treatment of this type, the internal material stresses which have been introduced into the material upon deformation of the cut-out blank into the form of the constrictor, will generally also be reduced or removed.

The constrictor which is thus obtained, such as the constrictor portrayed in FIGS. 1-4, is in the so-called first state. In this first state, the constrictor is preferably substantially slack, that is to say free from tension.

From this first state, the constrictor 1 according to the invention can be deformed (transformed) into a second state, in which the constrictor is under a pretension acting in the direction of the first state. That is to say, the pretension which is present in the constrictor in the second state is inclined to want to return the clamp to the first state. This pretension can be seen as a sort of resilient reaction force in reaction to the deformation of the constrictor from the first state into the second state. In the case of a spring-steel-like resilient material, this pretension will be palpable directly upon deformation and will demand a physical obstruction in order to be able to keep the constrictor in the second state.

For the production of the constrictor, according to the invention in particular a shape memory alloy is used. According to the invention, such a shape memory alloy can be a nickel-titanium alloy (NiTi alloy), also known as nitinol, a copper-aluminium-nickel alloy (CuAlNi alloy), a copper-zinc-aluminium alloy (CuZiAl alloy), or some other shape memory alloy. A shape memory alloy used to produce a constrictor according to the invention can also, however, be a (ferro)magnetic shape memory alloy. The terms ferromagnetic and magnetic are in practice used interchangeably. An example of a freely known (ferro)magnetic shape memory alloy of this type is a Ni₂MnGa-alloy. In place of a shape memory alloy, a plastics material, such as a memory elastomer, can also be used according to the invention.

If the constrictor is made of such a shape memory material, it will be able to be deformed from the first state into the second state and be able to remain in this second state without a physical obstruction being necessary to keep the constrictor in the second state, at least as long as the temperature remains below the temperature required for release or as long as no magnetic field is applied. Where a shape memory material is used, the constrictor will want to return from the second state into the first state only once the memory effect is activated. Activation of the memory effect is generally realized by heating the constrictor to above a certain threshold temperature (or possibly cooling it to below a certain threshold temperature) or by subjecting the constrictor to a magnetic field. Upon activation of the shape memory material, the pretension present in the memory is released in a manner comparable with that of removing a physical obstruction in the case of a material of spring-steel-like resilience.

The constrictor according to the invention can be deformed into the second state. Two different deformations can hereupon take place, which preferably occur in combination. The first deformation concerns the bringing of the pins 4, 5 towards an axially directed state. Preferably, the axially directed state is hereupon also reached, but it is here a matter, in particular, of the deformation of the pins in the direction of an axially directed state. Where the pins 4, 5 are curved, this will generally go hand in hand with a stretching of the pins, whereby they will acquire a stretched shape, or at least a less curved shape. The second deformation concerns the enlargement of the diameter of the ring 2.

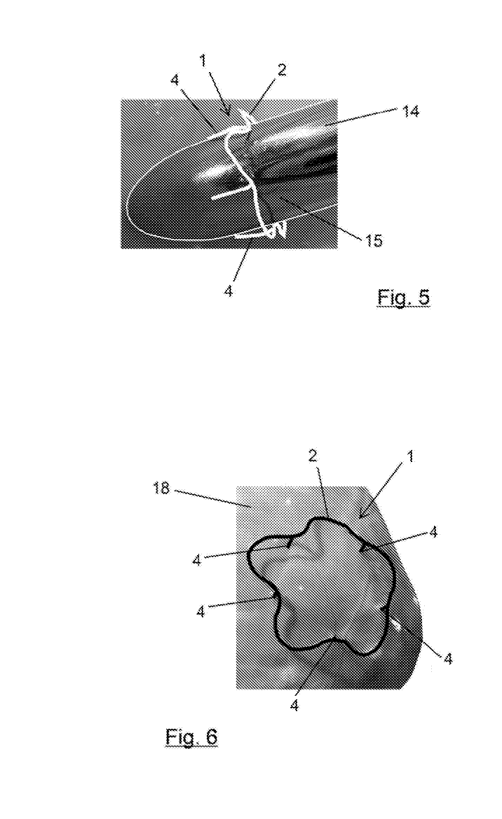

FIG. 5 shows a photo of the constrictor according to FIGS. 1-4 in a second state, in which both deformations have taken place and the deformed constrictor 1 has been provided, in this example, the cylindrical portion 15 of a cylindrical rod with rounded-off end, which rod here represents a medical instrument 14. The constrictor 1 and the contour of the medical instrument 14 are portrayed in the photo of FIG. 5 in white lines, for the purpose of having sufficient contrast against the dark background. In FIGS. 6-10, the constrictor and contours are respectively denoted by black lines, since the background of the photo is lighter there. It is noted that on the cylindrical portion 15 of the medical instrument 14 can be provided a knobbly pattern, the knobs of which fit into the waves of the wavy pattern of the—diametrically stretched—ring. Unwanted rotation of the constrictor with respect to the cylindrical portion 15 can thus be prevented. Such a knobbly pattern can be of use where pins 4 or 5 are used, but is particularly useful where pins 4 and 5 are used.

When the pins 4 (and/or pins 5) are brought into the axially directed state—the first deformation—the pins enter into an approximately axial orientation. Where the pins are curved, a remnant of the curvature—with larger radius of curvature—can remain, as can be seen from the lowermost pin 4 in FIG. 5.

When the pins 4—and/or pins 5—are brought into the axially directed state, three effects arise, which each result in a pretension in the constrictor which can later be released:
The first effect is that the trough portion 17 of the wave pattern, where the pin 4 (and/or pin 5) is rigidly attached to the ring 2, is subjected to a torsion. In reaction to this torsion, a torsional stress is built up in the shape memory alloy, which torsional stress can later be activated/released by subjection to, for example, heat or a magnetic field, or by removal of a mechanical obstruction. The pretension is then released, as it were, in order to pivot the pin back towards the position associated with the first state under the influence of the torsional stress.
The second effect is that the pin 4 (and/or pin 5)—upon activation/release by, for example, heat or a magnetic field or removal of a mechanical obstruction—as a result of bending stresses stored herein, will again want to revert to its (more) curved position associated with the first state. This second effect will especially occur if the particular pin, in the first state, is curved.
The third effect is that, as a result of the pins 4 (and/or pins 5) being brought into the axially directed state, the ring plane 8 will tilt with respect to the axial centre line 9, that is to say the ring plane angle β changes. The outer side 6 of the ring plane 8 is displaced with respect to the inner side 7 of the ring plane in the axial direction. In FIG. 2, this is a displacement of the outer side 6 in the upward direction with respect to the inner side 7, so that in this case the ring plane 8 is tilted into a flatter position, in which the height H, viewed in the axial direction—see FIG. 2—of the ring plane is smaller. The reaction stresses introduced into the ring as a result of this tilting of the ring plane will, upon release/activation, result in a tilting back of the ring, which acts also in the radially inward direction upon the pins 4. This tilting back of the ring into the earlier first state also has the result that the peak portions 16 of the wave pattern are pressed more firmly into the underlying tissue and are inclined to push this tissue in the direction of the axial centre line. It is noted that the ring plane angle β can also change from an angle less than 90° (first state) to an angle greater than 90° (second state); as well as that the ring plane angle β in the first state can already be greater than 90°, so as to increase still further during passage towards the second state.

Upon activation/release from the axially directed state, also termed the stretched state, the pin 4 is thus pressed inwards by a) pretension, stored in the pin, in the form of bending stresses which are inclined to push the pin towards its more curved position, by b) the pretension, stored in the trough portion, in the form of torsional stresses which are inclined to pivot the pin by its free end 13 towards the axial centre line, and by c) the return of the ring plane 8 of the ring 2 to its more oblique, less flat position. When the pins 4, in the stretched state, are stuck in tissue, already these effects help to ensure that the tissue located in-between the pins is pressed towards the axial centre line 9 of the ring. As has already several times been indicated above by the use of “and/or pin 5”, the same applies to the pins 5, which can be used in place of the pins 4 or can be used in combination with the pins 4. With regard to the pins 5—which are attached to the peaks 16 of the wave pattern—it is noted that, on the one hand, the above-described third effect additionally helps to ensure that the pins 5 will be able to be pressed more firmly into the tissue and that, on the other hand, also a fourth effect can arise. This fourth effect is that, viewed in the radial direction of the ring plane, the curvature of the ring plane can change. Changing of the curvature of the ring plane 8 will give rise to bending stresses in the flanks 31 of the wavy pattern. Upon release/activation, this will result in one and the same effect as results from the third effect upon release/activation. This fourth effect can also be generated free from the presence of pins 5 by, when the constrictor is deformed from the first into the second state, changing the curvature of the ring plane, viewed in the radial direction of the ring.

With reference to the pins 33 as portrayed in FIG. 3, it is noted that the previously discussed first effect and second effect will clearly also occur. If present in the ring, the third effect and the fourth effect, as well as the reduction in the diameter of the ring, will here too result in the pins 33 being able to apply to the tissue a force acting towards the centre of the ring.

When the diameter of the ring 2 is enlarged, the second deformation, the wavelengths of the waves of the wavy pattern—viewed in the peripheral direction of the ring—increase, whilst the amplitudes of the wave pattern—viewed transversely to these wavelengths—decrease. The troughs 17 and peaks 16 of the wavy pattern hereupon flatten off. Bending stresses are hereby introduced into the ring, inter alia into the peaks and troughs of the wave pattern thereof, which bending stresses, following their release, such as by activation with heat or a magnetic field, are inclined to make the wave pattern return to its form with smaller diameter, associated with the first state. The result of this reduction in diameter is that tissue gripped by the pins 4, 5 is pressed in the direction of the axial centre line of the ring 2.

The previously described first deformation with its three or, in the case of pins 5, even four effects, as well as the second deformation, make the constrictor according to the invention eminently suitable for closing or constricting a passage through tissue. This can be a naturally present passage, a passage or widening created by disease, a passage formed by a surgeon, or a passage or widening in some other sense. In particular, the Inventor provides that the constrictor according to the invention can be very suitably used for closing a passage through the wall of a blood vessel—such as, for example, the feed opening formed for the introduction of a catheter into a blood vessel—or the opening, formed through the wall of a heart or some other hollow organ, via which instruments and or prostheses can be brought inward into the heart or other hollow organ for the purpose of an intervention or a diagnostic procedure. The constrictor can also be used to constrict or strengthen a natural passage, or a passage widened by disease, in a hollow organ, as can be the case, for example, with heart valves.

In the event of the complete closure of a passage, it can be of advantage if the area covered by the constrictor—that is to say the area 32 described by the trough centre line 7 and possibly also the ring plane 8—is spanned or sealed off with a material which is impermeable to fluid, such as blood. In this context, plastics which are tolerated by the (human) body, such as plastics commonly used for vascular prostheses, or endogenic or exogenic pericardium, which can be fastened to the ring, for example, with stitching wire, can be considered. By fastening this sealing material with an excessive surface area to the constrictor, it is possible to prevent this sealing material from obstructing the temporary second state of the constrictor.

FIGS. 6-10 show with reference to photos an example of application of the constrictor according to the invention for closing a passage formed through the cardiac wall.

FIG. 5 shows the constrictor 1 according to FIGS. 1-4 in the so-called second state. It can be seen that this constrictor 1 has been subjected to both previously discussed deformations. The diameter of the ring is enlarged and the pins 4 are stretched. Where the constrictor 1 is made of a shape memory alloy, this can by itself remain in the state shown in FIG. 5 until the tensions stored in the constrictor as a result of the deformation are released/activated by heat (or cold) or a magnetic field or by removal of a mechanical obstruction. In FIG. 5, the constrictor 1 is fitted on a medical instrument 14 which can be used to place the constrictor in tissue. To this end, the medical instrument can also be considerably adapted in order to be able to control and monitor the placement of the constrictor and delivery of the pin from the instrument 14 as efficiently and accurately as possible. It is also very conceivable to place the constrictor according to the invention, when this is in the second state, manually in tissue, without any instrument.

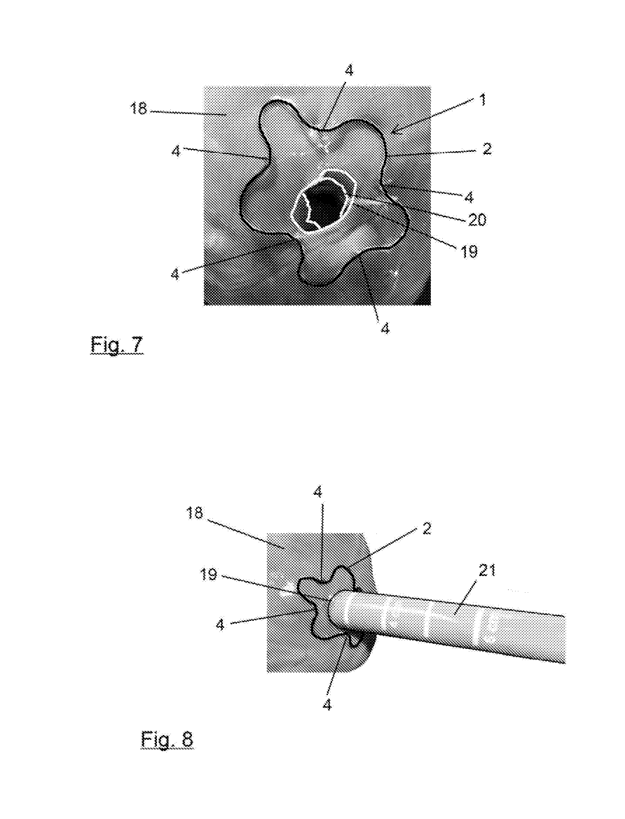

The instrument for placing the constrictor in the patient is advantageously designed such that the pins and the ring can be released separately in two stages. In this way, the pins 4,5 can first be pushed, for example, in the axial direction into the tissue, whereupon the pins will pivot in a ‘pre-programmed’ manner in the radially inward direction (the previously discussed first effect) and will bend (the previously discussed second effect), whilst the ring 2 is kept in the second state of enlarged diameter. Depending on how the instrument supports the ring, the tilting of the ring plane can also in this phase already be released (the previously discussed third effect), as can also the bending back of the ring plane (the previously discussed fourth effect which can occur where pins 5 are used). This then has the advantage that an instrument or guide wire can move freely in the axial direction through the constrictor 1 without the free ends of pins 4, and possibly also pins 5, obstructing this movement of the instrument, since they would then bear all too closely one against another at the site of this axial centre line. Only after the conclusion of the intervention is the ring 2 then released into the first state, wherein the passage through the tissue is closed off or constricted. This two-stage freeing of the clamp then has the advantage that, during the intervention, possible leakage of fluid, such as blood, for example, is prevented or reduced by the clamping effect of the pins. This can be realized, for example, with applicators, as portrayed in FIGS. 4, 8, 10 and 13 of PCT/NL2011/050202. In the applicators according to FIGS. 8 and 10, the distal drive part of the applicator should then be removed and turned round, as it were, so that this drive part acts from the proximal part of the applicator, for example as shown in FIG. 4 of PCT/NL2011/050202. In an applicator according to FIG. 13, the nose 1506, 1508 will then be absent, and the part 1504 will be of concave and flattened design to enable the applicator loaded with the constrictor to be placed with its flattened bottom edge of 1504 on and around a conical tissue structure. When the constrictor according to the invention is applied to valves or other natural passages, the applicators as portrayed in FIGS. 4, 8, 10 and 13 of PCT/NL2011/050202 can be used without modification or with minor modifications.

If we take as an example the use of the constrictor 1 as an apical closing device in a trans-apical aorta catheter valve (TAVI), then a two-stage procedure of this type could appear as follows. The constrictor 1 is cooled in, for example, ice water of 4-10 degrees C., and the ring 2 is placed in extended state with stretched pins 4,5 onto the applicator. Next the applicator is placed onto the apex of the heart and the pins 4,5 are pressed into the heart muscle, whereafter they bend over. In advance of or after this, a guide wire is put through the centre of the ring 2, over which a dilator and the valve balloon and the catheter valve are placed or positioned. After the conclusion of the intervention, the guide wire is removed and also the ring 2 released, whereby the passage through the heart muscle is totally closed off.

FIG. 6 shows the constrictor, currently in the second state, from FIGS. 1-5, which constrictor has been pressed by its legs 4, from outside, into a heart—of in this case a pig. FIG. 7 shows that after this, in the centre of the region surrounded by the ring 2, a passage 19, 20—see the white lines in the photo—through the wall of the heart into a heart chamber has been formed. 19 here denotes the top edge on the outer side of the passage and 20 denotes the bottom edge on the inner side, where the passage opens out into a heart chamber. As can be seen in FIG. 8, an instrument 21 having a thickness of 1 to 1.5 cm can comfortably be placed inwards through the passage 19, 20. Once the instrument 21 is removed, the passage 19, 20 can be closed by activation of the constrictor 1 by subjecting this to heat. Having been activated, the constrictor 1 will want to revert towards the first state. Whether this first state is also actually totally reached is dependent on factors such as the characteristics of the tissue, such as thickness and condition of the tissue. Moreover, it offers an advantage if the (original) state is not exactly fully regained. In that case, the constrictor will namely continue to apply to the tissue gripped by the constrictor a force which is directed towards the axial centre line.

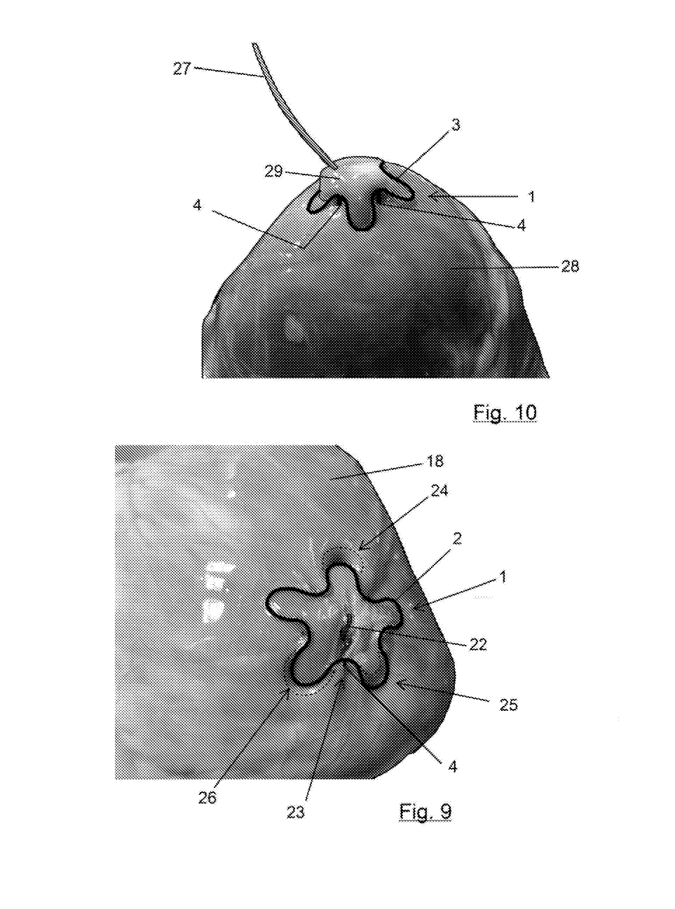

FIG. 9 shows the state in which the passage 19, 20 from FIG. 7-8 has been closed again by the constrictor. The closed passage is denoted by 22. It can be seen that the wave pattern, as regards wavelength and amplitude, has again, unsprung, assumed the state as shown in FIGS. 1-4. This also applies to the pins 4, which are barely visible in FIG. 9 since they are substantially stuck into the tissue. In FIG. 9 it can further be seen—emphasized with a thin dashed line—that three of the five wave lobes are clearly pressing into the tissue, see the wave lobes denoted by arrows 24, 25 and 26. This contributes to the closing effect of the constrictor 1 in the pinching-off of the passage 22.

FIG. 10 shows also by way of example a photo of another experiment on a pig's heart 28. The constrictor 1 is here activated in order to return to the first state and pushes tissue enclosed by the ring 2 of the constrictor 1 up to form a bulge 29, thereby helping to fully close off the passage. FIG. 10 further shows a guide wire 27, which, via a passage formed in the bulge and pinched-off, moreover, by the ring, has been stuck into the heart.

The first constrictor from FIGS. 1-10, the second constrictor from FIGS. 11-13 and the third constrictor from FIG. 14 can have the same dimensions or different dimensions. With reference to FIGS. 12 and 13 of the second constrictor 102, by way of example and viewed in the first state, a few dimensions are given here: diameter trough centre line 7 about 15 mm; diameter peak centre line 6 about 28.2 mm; radius R₁ about 3 mm; radius R₂ about 3 mm; width S₁ of the pins 4 and 5 about 0.5 mm; thickness S₂ of the pins 4 and 5 about 0.5 mm; height H of the constrictor about 10.3 mm; and length L of the pins 4 and 5 about 8.54 mm. Just as with the pins 4, 5, the width and thickness of the wire 3 are both about 0.5 mm. It is noted that these dimensions are simply intended as illustrative examples and are based on an experimental prototype. In practice, the dimensions, as well as other shaping, will be dependent on the intended application of the constrictor according to the invention. If the constrictor according to the invention is to be used as a constrictor for a widened heart valve, then, for example, the peak centre line 7 and trough centre line 6 will be able to have a larger diameter, whilst the lengths L of the pins 4 and/or 5 will be a bit shorter in order to prevent them from projecting in the radially inward direction on the inner side of the valve annulus.

The constrictor according to the invention makes it possible to conduct interventions in the innermost part of the heart via a passage formed through the cardiac wall. The heart can here be reached without the chest of the patient having to be opened up by means of, for example, a sternum spreader. The access to the heart is possible between two ribs by the removal or possible removal of a rib, whether partially or not.

The constrictor according to the invention can additionally very well be used in combination with a temporary work channel to a hollow organ, such as is described, inter alia, in earlier patent applications of the present Inventor, such as PCT/NL2011/050202 and WO-00/44311. In such a combination, even operations on a beating heart are possible.

1-44. (canceled)

45. A constrictor for closing or constricting a passage through tissue of a hollow organ, such as the heart or a blood vessel,
wherein the constrictor comprises:
a ring consisting of a thread-like body, which, viewed in the peripheral direction of the ring, extends in a wavy pattern; and
pins for fastening to tissue surrounding the passage to be closed or constricted; which pins are arranged distributed over the periphery of the ring;
wherein the ring has:
a peak centre line, which is defined as the imaginary line on which the peaks of the waves of the wavy pattern lie;
a trough centre line, which is defined as the imaginary line on which the troughs of the waves of the wavy pattern lie;
a ring plane, which is defined as the plane which connects the peak centre line and trough centre line and in which the wavy pattern extends;
an axial centre line, which extends in the axial direction of the ring; and
a radial direction, which extends transversely to the axial centre line;
wherein the ring plane has a first side and a second side lying opposite the first side;
wherein each pin has a fixed end, which is rigidly attached to the ring, and has a free end, which is of pointed design;
wherein the constrictor is deformable from a first state into a second state, during which deformation a pretension builds up such that the constrictor, in the second state, is under a pretension acting in the direction of the first state; and
wherein:
the pretension, in this second state, comprises a torsional stress which is present in portions of the thread-like body at the fixed end of each pin, which torsional stress is inclined to pivot the respective pin with respect to the ring back into that position of the respective pin with respect to the ring which is associated with the first state;
and/or
the thread-like body, in portions thereof at the fixed end of each pin, is twisted.

46. The constrictor according to claim 45,
wherein the ring plane, in the first state and in the second state, extends in the radial direction;
wherein, in the first state, the free ends of the pins point in the direction of the axial centre line; and
wherein, in the second state, the free ends of the pins point in the axial direction of the ring.

47. The constrictor according to claim 45, wherein, in the second state, the torsional stress is directed such that it moves the free end of the respective pin in the direction of the axial centre line of the ring or moves it away from the axial centre line of the ring.

48. The constrictor according to claim 45, wherein the constrictor is made of a shape memory material, such as a shape memory elastomer or a shape memory alloy, for example a nickel-titanium alloy.

49. The constrictor according to claim 45, wherein the constrictor, in the first state, is slack.

50. The constrictor according to claim 45, wherein the pins are attached to:
the, with respect to the ring, inwardly facing troughs of the waves of the wavy pattern, in particular in the middle of these troughs;
and/or
the, with respect to the ring, outwardly facing peaks of the waves of the wavy pattern, in particular in the middle of these peaks;
and/or
flanks of the waves of the wavy pattern, in particular in the middle of these flanks;
and wherein those portions of the thread-like body which in the second state are under torsional stress are respectively the troughs, and/or the peaks and/or the flanks.

51. The constrictor according to claim 45, wherein, viewed and measured from that portion of the axial centre line which is located on the first side of the ring plane, the ring plane angle which the ring plane exhibits with respect to the axial centre line is greater in the second state than in the first state.

52. The constrictor according to claim 45, wherein the pins, in the first state and in the second state, are located on the first side of the ring plane.

53. The constrictor according to claim 51, wherein the pins, in the first state and in the second state, are located on the first side of the ring plane.

54. The constrictor according to claim 45, wherein the ring is flatter in the second state than in the first state.

55. The constrictor according to claim 51, wherein the ring is flatter in the second state than in the first state.

56. The constrictor according to claim 45, wherein the diameter of the ring is greater in the second state than in the first state.

57. The constrictor according to claim 45, wherein the pretension, in this second state, comprises a bending stress which is present in the pins, which bending stress is inclined to bend the respective pin such that the free end of the pin moves towards the centre of the ring.

58. The constrictor according to claim 45, wherein the pins, in the second state, are stretched in the axial direction.

59. The constrictor according to claim 45, wherein the pretension, in this second state, comprises a bending stress which is present in the flanks of the wavy thread-like body, which bending stress is inclined to bend the respective flank such that the peaks of the wavy pattern move towards the axial centre line.

60. The constrictor according to claim 45, wherein the free ends of the pins, in the first state, lie close to the axial centre line of the ring, such as at 5 mm or less from the axial centre line.

61. The constrictor according to claim 45, wherein, in the first state:
the ring plane extends in the radial direction; and
the pins are arc-shaped with an arc angle of at least 20°, such as at least 30°, and extend in a plane transversely to the ring plane.

62. The constrictor according to claim 45, wherein, in the first state, the arc shape of the pins extends over an arc angle of at least 60°, such as at least 75° or about 90°.

63. The constrictor according to claim 45, wherein, in the second state, the pins are stretched in an arc shape reduced with respect to the first state and extend in the axial direction.

64. The constrictor according to claim 62, wherein, in the second state, the pins are stretched in an arc shape reduced with respect to the first state and extend in the axial direction.

65. The constrictor according to claim 45, wherein, viewed on this first side and in the first state, the ring plane extends at a ring plane angle of 30° to 80°, such as 45° to 80° or 45° to 70°, with respect to the axial centre line.

66. The constrictor according to claim 45, wherein, viewed on this first side and in the second state, the ring plane extends at a ring plane angle with respect to the axial centre line, which is at least 10°, such as 15° to 45° or 15° to 30°, greater than the ring plane angle which the ring plane, viewed on the first side and in the first state, exhibits with respect to the axial centre line.

67. The constrictor according to claim 45, wherein, viewed on the first side and in the second state, the ring plane extends at a ring plane angle of 45° to 120° with respect to the axial centre line.

68. The constrictor according to claim 45, wherein, in the first state, the ring plane has a conical shape or the shape of a portion of a cylinder.

69. The constrictor according to claim 45, wherein the ring and pins are formed as a complete whole by cutting-out:
from a single plate, in particular a flat plate; or
from a single three-dimensional body.

70. The constrictor according to claim 45, wherein the thread-like body and the pins have a right-angled cross section.

71. The constrictor according to claim 45, wherein the external diameter of the ring, in the first state, is less than or equal to 30 mm, such as less than or equal to 20 mm.

72. The constrictor according to claim 45, wherein the constrictor is in the second state.

73. An assembly comprising:
a constrictor according to claim 45; and
a medical instrument;
wherein the medical instrument comprises a pin-shaped portion, on which the constrictor, currently in the second state, is provided.

74. The assembly according to claim 73, wherein the pin-shaped portion is provided on the outer periphery thereof with a knobbed pattern, whereof the knobs fit in the waves of the wavy pattern of the ring.

75. A method for producing a constrictor according to claim 45, comprising the following steps:
the cutting-out of the ring, consisting of a thread-like body and pins, as a complete whole from a plate of a shape memory material, wherein the pins point with their free ends, viewed with respect to the ring, in a radially outward direction;
the bringing of the cut-out ring with pins into a first state, in which the pins lie with their free ends on the first side of the ring plane, point in the direction of the axial centre line and extend in a plane transversely to the ring plane defined by the ring;
the subjection of the constrictor, currently in a first state, to a temperature treatment, such that this first state is stored in the memory of the shape memory alloy.

76. The method according to claim 75, wherein the step of bringing the ring with pins into a first state comprises: the pivoting of the pins into a position in which the free ends thereof point towards the axial centre line of the ring, such that the thread-like body at the fixed end of each respective pin twists.

77. The method according to claim 75, wherein the step of bringing the ring with pins into a first state comprises: the curving of the pins into an arc shape with an arc angle of at least 30°.

78. The method according to claim 75, wherein the wavy pattern is formed during the cutting-out step by cutting out the ring from the plate in accordance with that wave pattern.

79. The method according to claim 75, wherein the wavy pattern is formed, during the step of the bringing into the first state, by deforming the thread-like ring into the wavy pattern.

80. The method according to claim 75, wherein the wavy pattern of the ring, during the step of the bringing into the first state, is formed into a structure having a conical shape or having the shape of a portion of a cylinder.

81. The method for preparing for use a constrictor according to claim 45, wherein the constrictor is brought from the first state into the second state by pivoting the pins such that in the ring, at the fixed end of each pin, a torsional stress builds up, which torsional stress acts in a direction with the intent of pivoting the pin with respect to the ring back in the direction of that position of the respective pin which is associated with the first state.

82. The method according to claim 81, wherein the pins, in the conversion from the first state into the second state, are pivoted into a position which is oriented axially with respect to the ring.

83. The method according to claim 81, wherein the pins, when the constrictor is brought from the first state into the second state, are bent, so that in each pin a bending stress builds up, which bending stress acts with the intent of bending the pin back into that shape of the respective pin which is associated with the first state.

84. The method according to claim 81, wherein, when the constrictor is brought from the first state into the second state, the ring plane angle which the ring plane exhibits with respect to the axial centre line is changed.

85. The method according to claim 81, wherein, when the constrictor is brought from the first state into the second state, the ring plane angle which the ring plane exhibits with respect to the axial centre line changes as a result of the pivoting of the pins.

86. The method according to claim 81, wherein, when the constrictor is brought from the first state into the second state, and viewed in the radial direction of the ring plane, the curvature of the ring plane, which the ring plane exhibits with respect to the axial centre line, is changed.

87. The method according to claim 81, wherein, when the constrictor is brought from the first state into the second state, and viewed in the radial direction of the ring plane, that curvature of the ring plane, which the ring plane exhibits with respect to the axial centre line, changes as a result of the pivoting of the pins.

88. The method according to claim 81, wherein, when the constrictor is brought from the first state into the second state, the diameter of the ring is enlarged.

89. The method for placing a constrictor according to claim 45 in tissue,
wherein in a first step, from the second state with enlarged diameter of the ring and with pins stretched in the axial direction, the pins are stuck into the tissue and are released in order to return in the direction of that position of the pins which is associated with the first state, whilst the ring is refrained from returning to the form associated with the first state;
wherein in a second step the ring is released in order to return towards the form associated with the first state; and
wherein the second step takes place at a later point than the first step.
